(19) United States
(12) Patent Application Publication
(10) Pub. No.: US 2005/0255466 A1
(43) Pub. Date: Nov. 17, 2005

(54) Method and system for determining absolute mrna quantities

(75) Inventors: Benedikt J Brors, Heidelberg (DE); Nicole Hauser, Heidelberg (DE); Martin Vingron, Berlin (DE)

Correspondence Address: FISH & NEAVE IP GROUP;ROPES & GRAY LLP, ONE INTERNATIONAL PLACE, BOSTON, MA, 02110-2624, US

(73) Assignee: Deutsches Krebsforschungszntrum, Heidelberg (DE)

(21) Appl. No.: 10/509,275

(22) PCT Filed: Mar. 28, 2003
(86) PCT No.: PCT/EP03/03291

§ 371 Date: May 25, 2005

(30) Foreign Application Priority Data
Mar. 28, 2002 (EP) 02007267.4

(51) Int. Cl. C12Q 1/68

(52) U.S. Cl. 435/6

(57) ABSTRACT

The present invention is particularly directed to a method and a system for determining absolute mRNA quantities, specifically on cDNA microarrays. The following steps and respective means are provided: providing a microarray; providing at least one or more dilution series of control spots on this microarray; hybridizing with a corresponding control cDNA of known concentration; deriving reference data from the hybridization and using said reference data to calculate absolute mRNA concentrations in one or more samples used.

The invention is directed to a method and a system for determining absolute mRNA quantities by means of spotted cDNA microarrays.

The use of DNA microarrays has revolutionized the investigation of gene expression due to its ability to measure mRNA quantities for thousands of different transcripts at the same time¹⁻¹⁶. Briefly, representative single stranded DNA fragments are immobilized on a solid support (e.g. a glass slide) to probe for complementary cDNAs or cRNAs. These are prepared from biological samples as a representation of their mRNA pool. The preparation involves reverse transcription, and—in the case of cRNA—additional in-vitro-transcription as a means for amplification. Complementary DNAs or cRNAs are concomitantly labeled by incorporation of radioactive isotopes or of nucleotides that have been modified by attaching a fluorescent dye. After hybridization to the microarray, weakly or unspecific bound cDNAs or cRNAs are washed away, and the signal is read out by a phosphorimager (for radioactive label) or a confocal laser scanning device (for fluorescent label). Computational image analysis determines for each feature (called “spot”) on the array its position (and thereby its identity) and the signal intensity, which is believed to be linearly related to the original amount of cDNA or cRNA of that species that is represented by this feature.

With DNA microarrays, it has become possible to monitor changes in mRNA levels for thousands of different molecules in a single experiment. The technique of “spotted” cDNA chips has found widespread use, but allows currently to measure only relative changes of mRNA concentration in a cell. Moreover, due to technical limitations in the manufacturing process, results can only be made comparable for chips that stem from the same production series, i.e. experiments are currently limited to approximately 50-200 hybridizations.

There are still a number of technical problems that have to be corrected for in later data analysis. The most prominent problem is normalization, or standardization, which accounts for differences in labeling efficiency and mRNA amount between two samples whose cDNA representation is hybridized to two different chips, or to the same chip but labeled with fluorescent tags that light up in different colors when excited by UV laser light, and can thus be distinguished¹⁷⁻²⁵.

The object underlying the present invention is to provide an improved method and system for determining absolute mRNA quantities.

This object is achieved with the subject-matter as recited in the claims.

The invention provides a method to overcome these technical limitations and measure absolute mRNA quantities, which are then comparable independent of the chip series used. The method involves a dilution series of control spots on the microarray and hybridization with a corresponding control DNA/cDNA of known concentration. According to a preferred embodiment a model curve is then fitted to the obtained signals for the control, and can be used to calculate absolute mRNA concentrations in the sample used. Since these measurements are done with cDNA microarrays, they are able to incorporate as many as 25,000 different mRNA species in a single hybridization experiment.

Another problem that could not be solved yet is that chips that have been produced by “spotting” display high batch-to-batch variation in the surface concentration of DNA fragments in individual spots. In this method, cDNA templates for the genes that are to be probed for are first amplified by large-scale multiplex polymerase chain reaction (PCR). The amplified fragments are than transferred to the microarray supports (mostly glass chips with chemically modified surfaces) by means of roboting devices, which are able to deliver nanoliter quantities with a spatial precision of better than 100 μm. Recently, ink jet-like printing devices have been used for the same purpose²⁶. In any case, once the produced PCR fragments are exhausted, a new PCR preparation has to be started from the master templates. It is currently impossible to control the yield of the PCR reaction for thousands of different templates at the same time. DNA concentration can be measured thereafter, however, transfer rates and coupling efficiency cannot be captured by this means. Thus, spotting microarrays in different batch results in chips that vary in individual spot DNA concentration by factors of up to 100 compared with a previous microarray production series. The influence of the DNA spot concentration on recorded signal intensity after hybridization is immediately obvious, and cannot be overcome by competitive hybridization where only a ratio of transcript levels is measured for two samples, one whose cDNA representation serves as internal reference, and one whose mRNA pool is to be investigated. The incomparability has been conclusively demonstrated by Yue and coworkers²⁵, who showed that this ratio is considerably dependent on the DNA spot concentration, and will be the more “compressed” (nearer to 1) the smaller the DNA spot concentration is.

Here, we present a novel method that allows a subsequent computational correction for the influence of the individual DNA spot concentration. It requires dilution series of control DNAs to be present on the array and several hybridizations with a universal target that binds to every spot on the array in defined concentrations. By fitting a model curve to the data obtained from the hybridization with the universal target, a subset of spots can be identified whose corresponding values need correction, whereas for the remaining spots correction would do more harm than benefit. The method extends current microarray applications in two directions. First, it is now possible to measure absolute quantities of different mRNAs by microarrays. Second, and more importantly, it is now possible to compare experiments done on different series of chips, and moreover also between different transcript level measuring systems like cDNA chips, oligonucleotide chips^{10,11}, SAGE (serial analysis of gene expression)^{27,28}, and multiplex real-time reverse transcription (RT) PCR^{29, 30}. This extends the “horizon of comparability”, which is currently limited to about 50-200 chips, to the levels that are required for new applications that require investigation of up to 500 samples, e.g. classification of cancer subtypes by means of gene expression profiling.

Throughout this text, the following terms will be used:
array, microarray, chip: a solid support (e.g. chemically modified glass or nylon/polypropylene membrane) that contains a number of features (spots), each containing a single species of DNA fragments
feature, spot: a single location on a microarray where only one species of DNA fragments is bound
hybridization: the process by which two complementary DNA molecules bind to each other, being driven by Watson-Crick base pairing
probe: one of the array-bound DNA fragments that are meant to probe for a specific transcript
(solute) target: the cDNA or cRNA preparation from a biological sample, intended to be the representation of the sample's mRNA pool
signal intensity: the intensity of radioactive radiation or the fluorescence intensity during laser excitation that has been read out for a single spot
(biological) sample: a specimen from the organism under study, derived under defined conditions; e.g., a sample from yeast culture, cell culture, a tumor specimen, a biopsy sample, a blood sample
universal target: a DNA fragment of defined sequence that binds to every spot on an array, e.g. a universal oligonucleotide

The figures relate to preferred embodiments which exemplify the present invention and show the following:

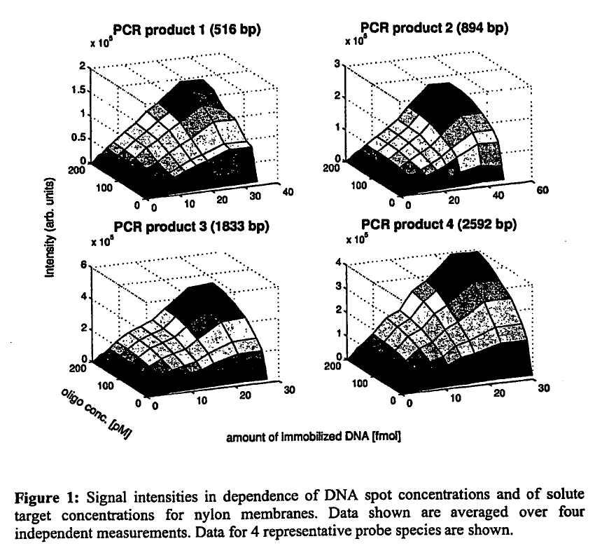

FIG. 1: Signal intensities in dependence of DNA spot concentrations and of solute target concentrations for nylon membranes. Data shown are averaged over four independent measurements. Data for 4 representative probe species are shown.

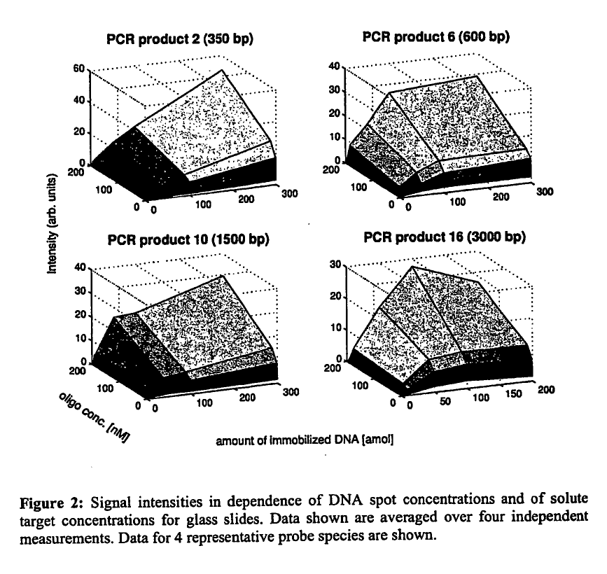

FIG. 2: Signal intensities in dependence of DNA spot concentrations and of solute target concentrations for glass slides. Data shown are averaged over four independent measurements. Data for 4 representative probe species are shown.

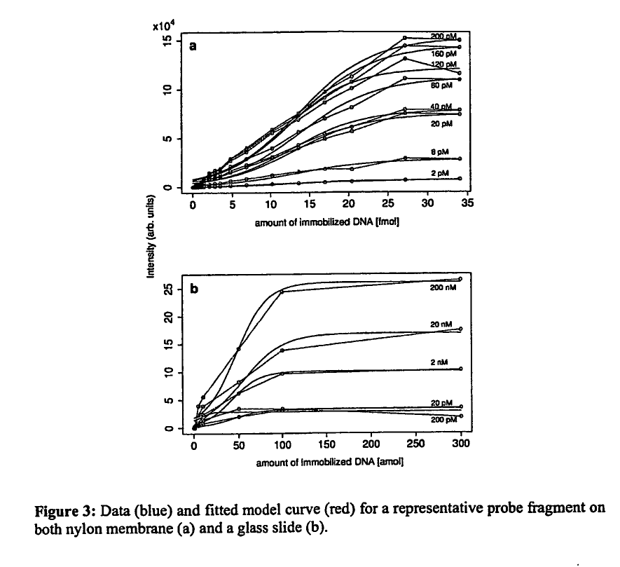

FIG. 3: Data (blue) and fitted model curve (red) for a representative probe fragment on both nylon membrane (a) and a glass slide (b).

### COMPUTATIONAL PROCEDURES

The values obtained from the hybridization to a universal target, i.e. the read-out signal intensities from the dilution series spots, are taken as a basis for calculating the parameters of a model function by means of non-linear least-squares fitting. As model function, we use the logistic function:

$$\hat{I} = \frac{K {I}_{0} {ⅇ}^{{rc}_{p}}}{K + {I}_{0} ( {ⅇ}^{{rc}_{p}} - 1 )} \qquad ( 1 )$$

where Î·refers to the modeled signal intensity, cₚ refers to the probe (or DNA spot) concentration, K represents the asymptotic signal intensity for cₚ→∞, I₀ is the asymptotic signal intensity for cₚ→0, and r is a shape parameter. We could demonstrate (see Results) that r does neither depend on the concentration of the solute target cDNA nor on the nature of the probe sequence, and hence should be the same for every spot on the array. Fitting is done by standard gradient optimization procedures for non-linear fitting, we used the Newton-Raphson method as implemented in MATLAB™ (MathWorks Inc., Natick, Mass., U.S.A.). The confidence of the fitting procedure is greatly enhanced by using several replicates of the dilution series on the chip, we recommend to use at least five.

The model function is used to determine the set of spots whose values need correction for the influence of spot DNA concentration. A critical probe concentration is defined by
c_{crit}=cₚ|Î=0.85K (2)
or, analytically

$${c}_{crit} = \frac{1}{r} [ ln \frac{17 ( K - {I}_{0} )}{3 {I}_{0}} ] \qquad ( 3 )$$

A hybridization with a universal target will provide for an array of a given series both the possibility to calculate the parameters of the model function, based on the values from the dilution series, and to determine for every spot in the array whether or not its spot DNA concentration is below or above the critical probe concentration. If it is above, no correction is needed since the probe concentration does not influence signal intensity. If it is below, the signal intensity is, in first approximation, linearly dependent on the spot DNA concentration, and values in later hybridizations can be corrected by taking the determined probe concentration from the hybridization with the universal target.

Experiments on Nylon and Polypropylene Membranes

As probe sequences, 16 different PCR fragments from yeast ORFs (Tab. 3) have been spotted in 9 different concentrations (Tab. 1), on both nylon and polypropylene membranes. These arrays have been hybridized with a universal oligonucleotide in 8 different concentrations (Tab. 2). Hybridization experiments have been carried out in quadruplicate, and signal intensities subsequently averaged.

TABLE 1
spot DNA amounts used
amount of immobilized DNA

| | | | | | | | | |
|---|---|---|---|---|---|---|---|---|
| 10 | 50 | 100 | 500 | 1 | 5 | 10 | 25 | 50 |
| amol | amol | amol | amol | fmol | fmol | fmol | fmol | fmol |

TABLE 2
target concentrations tested
concentration of target (radioactively labeled universal oligonucleotide)

| | | | | | | | |
|---|---|---|---|---|---|---|---|
| 2 pM | 8 pM | 20 pM | 40 pM | 80 pM | 120 pM | 160 pM | 200 pM |

TABLE 3
probe sequences used

| ORF identifier | length [bp] |
|---|---|
| YAR030C | 342 |
| YAR043C | 350 |
| YCR025C | 411 |
| YCL022C | 516 |
| YAL055W | 543 |
| YAR037W | 600 |
| YCL031C | 894 |
| YCR034W | 1044 |
| YCR045C | 1476 |
| YCR024C | 1479 |
| YCR048W | 1833 |
| YBR280C | 1929 |
| YDR422C | 2592 |
| YCR030C | 2613 |
| YDR430C | 2790 |
| YAL035W | 3009 |

As is obvious from FIG. 1, the measured signal intensities depend in a non-linear fashion on both the DNA spot concentration and on the concentration of the solute target. The dependence on DNA spot concentration is best described by a approximately linear interval at low spot concentrations that is followed by a saturation region where no increase in signal intensity can be seen if the spot concentration is increased.

There are no differences in principal shape to the other probe species and to data from polypropylene membranes (data not shown).

Experiments on glass slides Poly-L-lysine-coated glass slides have been used as solid support. The 16 PCR fragments mentioned above (Tab. 3) have been spotted in quadruplicate onto these slides in 8 different concentrations (Tab. 4). The arrays have been hybridized with a fluorescently marked universal oligonucleotide in 5 different concentrations (Tab. 5)

TABLE 4
amount of spot DNA
amount of immobilized DNA

| | | | | | | | |
|---|---|---|---|---|---|---|---|
| 0.1 | 0.5 | 1 | 5 | 10 | 50 | 100 | 300 |
| amol | amol | amol | amol | amol | amol | amol | amol |

TABLE 5
solute target concentrations used
concentration of solute target

| | | | | |
|---|---|---|---|---|
| 20 pM | 200 pM | 2 nM | 20 nM | 200 nM |

Data are shown in FIG. 2, and represent averages over the 4 spots on a single slide and the 4 independent replicate hybridizations that have been performed. Apart from a coarser resolution with regard to high DNA spot concentrations and a higher variability of the data, no principal difference is obvious compared to the experiments on nylon or polypropylene membranes.

Fitting of model curve to data Equation (1) has been fitted to the data by means of non-linear optimization. Of main interest is here the estimate for the shape parameter r. This parameter describes the steepness of the curve and hence determines the critical spot concentration which is used to split the range of DNA spot concentrations in a non-influential section at high spot concentrations, and in a range where the signal depends on the spot concentration. The results are shown for representative probe fragments both for nylon membranes and for glass slides (FIG. 3). The model function fits the data well for the transition from influential to non-influential region, while deviating considerably in the region with low spot concentration. However, with regard to the intended purpose, a good fit in this region is not needed.

### REFERENCES

1. Case-Green, S. C., Mir, K. U., Pritchard, C. E. & Southern, E. M. Analysing genetic information with DNA arrays. Curr. Opin. Chem. Biol. 2, 404-410 (1998).
2. DeRisi, J. et al. Use of a cDNA microarray to analyse gene expression patterns in human cancer. Nat. Genet. 14, 457-460 (1996).
3. DeRisi, J. L., Iyer, V. R. & Brown, P. O. Exploring the metabolic and genetic control of gene expression on a genomic scale. Science 278, 680-686 (1997).
4. Duggan, D. J., Bittner, M., Chen, Y., Meltzer, P. & Trent, J. M. Expression profiling using cDNA microarrays. Nat. Genet. 21, 10-14 (1999).
5. Golub, T. R. et al. Molecular classification of cancer: class discovery and class prediction by gene expression monitoring. Science 286, 531-537 (1999).
6. Hegde, P. et al. A concise guide to cDNA microarray analysis. Biotechniques 29, 548-550, 552-554, 556 passim (2000).
7. Hughes, T. R. et al. Functional discovery via a compendium of expression profiles. Cell 102, 109-126 (2000).
8. Iyer, V. R. et al. The transcriptional program in the response of human fibroblasts to serum. Science 283, 83-87 (1999).
9. Khan, J., Bittner, M., Chen, Y., Meltzer, P. S. & Trent, J. M. DNA microarray technology: the anticipated impact on the study of human disease. Biochim. Biophys. Acta 1423, M17-M28 (1999).
10. Lipshutz, R. J., Fodor, S. P., Gingeras, T. R. & Lockhart, D. J. High density synthetic oligonucleotide arrays. Nat. Genet. 21, 20-24 (1999).
11. Lockhart, D. J. et al. Expression monitoring by hybridization to high-density oligonucleotide arrays. Nat. Biotechnol. 14, 1675-1680 (1996).
12. Lockhart, D. J. & Winzeler, E. A. Genomics, gene expression and DNA arrays. Nature 405, 827-836 (2000).
13. Schena, M. Genome analysis with gene expression microarrays. BioEssays 18, 427-431 (1996).
14. Schena, M., Shalon, D., Davis, R. W. & Brown, P. O. Quantitative monitoring of gene expression patterns with a complementary DNA microarray. Science 270, 467-470 (1995).
15. Shalon, D., Smith, S. J. & Brown, P. O. A DNA microarray system for analyzing complex DNA samples using two-color fluorescent probe hybridization. Genome Res. 6, 639-645 (1996).
16. Spellman, P. T. et al. Comprehensive identification of cell cycle-regulated genes of the yeast Saccharomyces cerevisiae by microarray hybridization. Mol. Biol. Cell 9, 3273-3297 (1998).
17. Beiβbarth, T. et al. Processing and quality control of DNA array hybridization data. Bioinformatics 16, 1014-1022 (2000).
18. Chudin, E. et al. Assessment of the relationship between signal intensities and transcript concentration for Affymetrix GeneChip® arrays. Genome Biol. 3, research 0005.0001-0005.0010 (2001).
19. Eickhoff, B., Korn, B., Schick, M., Poustka, A. & Bosch, J.v.d. Normalization of array hybridization experiments in differential gene expression analysis. Nucl. Acids Res. 27, e33 (1999).
20. Lee, M.-L. T., Kuo, F. C., Whitmore, G A. & Sklar, J. Importance of replication in microarray gene expression studies: statistical methods and evidence from repetitive cDNA hybridizations. Proc. Natl. Acad. Sci. U.S.A. 97, 9834-9839 (2000).
21. Newton, M. A., Kendziorski, C. M., Richmond, C. S., Blattner, F. R. & Tsui, K. W. On differential variability of expression ratios: improving statistical inference about gene expression changes from microarray data. J. Comput. Biol. 8, 37-52 (2001).
22. Chen, Y., Dougherty, E. R. & Bittner, M. Ratio-based decisions and the quantitative analysis of cDNA microarray images. J. Biomed. Opt. 2, 364-374 (1997).
23. Schadt, E. E., Li, C., Su, C. & Wong, W. H. Analyzing high-density oligonucleotide gene expression array data J. Cell. Biochem. 80, 192-202 (2000).
24. Schuchhardt, J. et al. Normalization strategies for cDNA microarrays. Nucl. Acids Res. 28, E47 (2000).
25. Yue, H. et al. An evaluation of the performance of cDNA microarrays for detecting changes in global mRNA expression. Nucl. Acids Res. 29, E41 (2001).
26. Hughes, T. R. et al. Expression profiling using microarrays fabricated by an ink-jet oligonucleotide synthesizer. Nat Biotechnol 19, 342-347 (2001).
27. Velculescu, V. E., Zhang, L., Vogelstein, B. & Kinzier, K. W. Serial analysis of gene expression. Science 270, 484-487 (1995).
28. Velculescu, V. E. et al. Analysis of human transcriptomes. Nat. Genet. 23, 387-388 (1999).
29. Bustin, SA. Absolute quantification of mRNA using real-time reverse transcription polymerase chain reaction assays. J Mol Endocrinol 25, 169-193. (2000).
30. Freeman, W. M., Walker, S. J. & Vrana, K. E. Quantitative RT-PCR: pitfalls and potential. Biotechniques 26, 112-122, 124-115. (1999).

1. Method for determining absolute mRNA quantities by means of DNA microarrays with the following steps:
a. providing a microarray;
b. providing at least one or more dilution series of control spots on the microarray;
c. hybridizing with a corresponding control DNA of known concentration;
d. deriving reference data from the hybridization and
e. using said reference data to calculate absolute mRNA concentrations in one or more samples used.

2. Method according to claim 1, further comprising the step of providing a universal tag at each of the immobilized probes on the array to be used for quantification.

3. Method according to claim 1 or 2, wherein a DNA microarray is a cDNA microarray and/or the control DNA is control cDNA.

4. Method according to any of the preceding claims, wherein the reference data comprise a model curve which is fitted or adapted to the obtained signals for control.

5. Method according to any one of the preceding claims, wherein cDNA templates for the genes that are to be probed for are first amplified by large-scale multiplex polymerase chain reaction (PCR) to obtain amplified fragments.

6. Method according to claim 5, wherein said amplified fragments are then transferred to the microarray by means of roboting devices which are able to deliver nanoliter quantities with a spatial precision of better than 100 μm.

7. Method according to any one of the preceding claims, wherein the reference data obtained from the hybridization, e.g., the read-out signal intensities from the dilution series spots, are taken as a basis for calculating the parameters of a model function by means of non-linear least-squares fitting.

8. Method according to claim 7, wherein for the model function the following function is used:
$$\hat{I} = \frac{K {I}_{0} {ⅇ}^{{rc}_{p}}}{K + {I}_{0} ( {ⅇ}^{{rc}_{p}} - 1 )}$$
where Î refers to the modeled signal intensity, cₚ refers to the probe (or DNA spot) concentration, K represents the asymptotic signal intensity for cₚ→∞, I₀ is the asymptotic signal intensity for cₚ→0, and r is a shape parameter.

9. Method according to any one of claims 4-8, wherein fitting of the reference data is done by gradient optimization procedures.

10. Method according to claim 9, wherein for non-linear fitting, the Newton-Raphson method is used.

11. Method according to any one of the preceding claims, wherein a critical probe function is used to determine the set of spots whose values need correction for the influence of spot DNA concentration, the critical probe concentration function being defined by
$${c}_{crit} = \frac{1}{r} [ ln \frac{17 ( K - {I}_{0} )}{3 {I}_{0}} ] .$$

12. Computer program product comprising program code means stored on a computer readable medium for performing the computable part of the method of at least one of the preceding claims when said program product is run on a computer.

13. System, particularly for performing the method of at least one of claims 1-11, comprising:
a. a microarray containing at least one or more dilution series of control spots;
b. means for hybridizing with a complementary control DNA of known concentration;
c. means for deriving reference data from said hybridization and
d. means for making use of said reference data for calculating absolute mRNA concentrations in one or more samples used.

14. System according to claim 13, further comprising means for providing a universal tag at each of the immobilized probes on the array to be used for quantification.

15. Use of a method according to at least one of claims 1-11, a computer program product according to claim 12 and/or a system according to at least one of claims 13 or 14 for determining absolute mRNA quantities on cDNA microarrays.

16. Method of absolute quantification of other biomolecules like DNA or proteins in a complex sample by means of methods according to any one of claims 1-11.
